(19) United States
(12) Patent Application Publication
(10) Pub. No.: US 2019/0055278 A1
(43) Pub. Date: Feb. 21, 2019

(54) PROCESS FOR THE PREPARATION OF GUANYLATE CYCLASE 2C AGONIST

(71) Applicant: CIPLA LIMITED, Mumbai (IN)

(72) Inventors: Dharmaraj Ramachandra RAO, Thane (West) (IN); Geena MALHOTRA, Mumbai (IN); Venkata Srinivas PULLELA, Bangalore (IN); Parag Shankar KURLE, Navi Mumbai (IN); Rakesh MENDHE, Panvel (IN)

(21) Appl. No.: 16/075,521

(22) PCT Filed: Feb. 3, 2017
(86) PCT No.: PCT/IN2017/050051

§ 371 Date: Aug. 3, 2018

(30) Foreign Application Priority Data
Feb. 3, 2016 (IN) 201621003858
Apr. 15, 2016 (IN) 201621013272

(51) Int. Cl. C07K 1/04; C07K 7/08

(52) U.S. Cl. CPC C07K 1/04; C07K 7/08

(57) ABSTRACT

The present invention relates to an improved process for the preparation of Linaclotide of Formula I. The process disclosed in the present invention is simple, economical and eco-friendly with reduced reaction times.

### FIELD OF THE INVENTION

The present invention relates to an improved process for the preparation of Linaclotide of Formula I.

### BACKGROUND OF THE INVENTION

Linaclotide (marketed under the trade name Linzess and Constella) is a peptide agonist of the guanylate cyclase 2C (GC-C). Guanylate cyclase C agonist refers to a transmembrane form of guanylate cyclase that acts locally on intestinal epithelial cells as the intestinal receptor for the heat-stable toxin (ST) peptides secreted by enteric bacteria. Guanylate cyclase C increases cGMP production which triggers a signal transduction cascade leading to increased fluid secretion and accelerated colonic transport. Guanylate cyclase C is also the receptor for the naturally occurring peptides guanylin and uroguanylin.

Linaclotide reduces activation of colonic sensory neurons, reducing pain; and activates colonic motor neurons, which increases smooth muscle contraction and thus promotes bowel movements. It was approved by the FDA in August 2012 for the treatment of chronic idiopathic constipation and irritable bowel syndrome with constipation (IBS-C) in adults.

Both Linaclotide and its active metabolite bind to GC-C and act locally on the luminal surface of the intestinal epithelium. Activation of GC-C results in an increase in both intracellular and extracellular concentrations of cyclic guanosine monophosphate (cGMP). Elevation in intracellular cGMP stimulates secretion of chloride and bicarbonate into the intestinal lumen, mainly through activation of the cystic fibrosis transmembrane conductance regulator (CFTR) ion channel, resulting in increased intestinal fluid and accelerated transit.

In animal models, Linaclotide has been shown to both accelerate GI transit and reduce intestinal pain. The Linaclotide-induced reduction in visceral pain in animals is thought to be mediated by increased extracellular cGMP, which was shown to decrease the activity of pain-sensing nerves.

Linaclotide is a peptide consisting of 14 amino acids. The sequence is H⁻Cys¹⁻Cys²⁻Glu³⁻Ty⁴⁻Cys⁵⁻Cys⁶⁻Asn⁷⁻Pro⁸⁻Ala⁹⁻Cys¹⁰⁻Thr¹¹⁻Gly¹²⁻Cys¹³⁻Tyr¹⁴⁻OH

This molecule is cyclical by forming three disulfide bridges: between Cys¹ and Cys⁶, between Cys² and Cys¹⁰, and between Cys⁵ and Cys¹³ structurally analogous to the diarrhea-causing, heat-stable enterotoxins produced by E. coli.

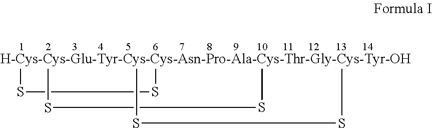

Linaclotide is marketed in USA under the trade name LINZESS in the form of capsules having dosage forms 145 meg and 290 meg for the treatment of irritable bowel syndrome with constipation and chronic idiopathic constipation.

Linaclotide was first disclosed in U.S. Pat. No. 7,304,036. This patent discloses two different methods for the preparation of Linaclotide either by solid phase synthesis or by recombinant DNA technology. Solid-phase synthesis is carried out by sequential addition of amino acids (Boc/Fmoc strategy) using an automated peptide synthesizer such as Cyc(4-CH2Bxl)-OCH2-4-(oxymethyl)-phenylacetamidomethyl resin to yield linear protected Linaclotide, which is deprotected and cleaved from resin using hydrogen fluoride, dimethyl sulfide, anisole and p-thiocresol. Thereafter obtained linear Linaclotide is oxidized, then purified using RP-HPLC and lyophilized to obtain Linaclotide in 10-20% yield.

Biopolymers, Issue 96, Volume 1, Pages 69-80 (2011) also discloses synthesis of Linaclotide by solid phase synthesis, following sequential addition of amino acids to the supported resin (Wang or 2-chlorotrityl resin) and thereafter cleaved from resin and de-protection is carried out in two steps. The above processes disclose synthesis of Linaclotide by sequential addition of amino acids to a solid support resin. The disadvantage of this process is that the final compound is obtained with inconsistent yields, because of premature loss of peptide during synthesis.

Preparation of Linaclotide with solid phase peptide synthesis is also disclosed in WO 2014/188011. The process involves preparation of Linaclotide by elongation with solid phase peptide synthesis, global deprotection and oxidation, followed by purification and drying.

WO 2012/118972 discloses a process for the preparation of Linaclotide by coupling the two fragments in solution phase in presence of a coupling agents HBTU, CI-HOBt, DIPEA, DMF to obtain linear protected Linaclotide, which is deprotected in presence of trifluoroacetic acid/ethanedithiol/triisopropylsilane/water (TFA:EDT:TIS:H₂O::85:5:5:5, v/v/v/v) and oxidation in presence of sodium bicarbonate and glutathione hydrochloride, followed by purification using preparative RP-HPLC and lyophilization.

The disadvantage of the above process is that the end product will be contaminated with many impurities which are difficult to remove later.

To overcome the drawbacks of this process one more process was disclosed in WO 2015/022575, which comprises steps of; a) preparing two or three suitable fragments by solid phase peptide synthesis; b) coupling of the fragments by solid phase synthesis to obtain a protected peptide; c) concurrent cleaving the protected peptide from the peptide resin and de-protecting the peptide; d) oxidizing the deprotected peptide to obtain Linaclotide; and e) isolation of Linaclotide. However, a drawback associated with the process of WO \check{} 575 is the use of different deprotecting agents, thereby introducing a large quantity of solvents in the process, which may affect the purity of the final polypeptide.

WO 2016/012938 discloses a process for preparing amorphous Linaclotide especially preparation of disulfide linkage by treating a linear chain of peptide at a maximum concentration of 13.9 mg/ml, with a suitable reagent to prepare appropriate disulfide bridges within linear chain of peptide. The crude Linaclotide is further purified by either anion/cation exchange chromatography or hydrophobic interaction followed by reverse-phase chromatography.

Thus there is continuous need in the art for new low-cost and high-yielding process for the preparation of Linaclotide suitable for industrial scale.

### Objective

The object of the present invention is to provide an improved process for the preparation of Linaclotide.

Another object of the present invention is to provide an improved process for preparing disulfide bridges of Linaclotide.

Another object of the invention is to provide a process for the preparation of Linaclotide which is simple, economical and eco-friendly with reduced reaction times.

### SUMMARY OF THE INVENTION

In a first aspect, the present invention provides a process for preparing Linaclotide of formula (I):

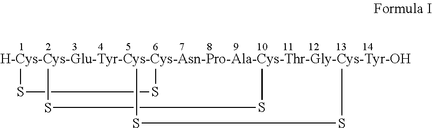

which comprises the following steps:

a) elongation of peptide with sequential addition of amino acids by solid phase synthesis to obtain a protected peptide resin;

b) cleaving and deprotecting the resin simultaneously to obtain the linear Linaclotide of Formula (II);

c) oxidizing the linear Linaclotide of Formula (II) to obtain Linaclotide of Formula (I); and

d) isolation of Linaclotide.

According to another aspect of the present invention, there is provided an improved process for preparing disulfide bridges of Linaclotide using higher concentration of linear Linaclotide.

According to another aspect of the present invention, there is provided Linaclotide prepared by the present process, having purity of more than 99% in a single purification step.

According to another aspect of the present invention, there is provided Linaclotide prepared according to the process of the present invention.

According to another aspect of the present invention, there is provided a pharmaceutical composition comprising Linaclotide prepared according to the process of the present invention together with one or more pharmaceutically acceptable excipients.

According to another aspect of the present invention, there is provided Linaclotide prepared according to the process of the present invention for use in treating irritable bowel syndrome and chronic constipation thereof.

According to another aspect of the present invention, there is provided a method of treating irritable bowel syndrome and chronic constipation thereof, the method comprising administering to a subject in need thereof a therapeutically effective amount of Linaclotide prepared according to the process of the present invention.

In another aspect, the present invention provides a process substantially as herein described with reference to the examples.

Further features of the present invention are defined in the dependent claims.

### BRIEF DESCRIPTION OF THE FIGURES

FIG. 1: Synthesis of Linaclotide using Fmoc-Cys(Trt)-OH, by elongation of peptide with sequential addition of amino acids using solid phase peptide synthesis

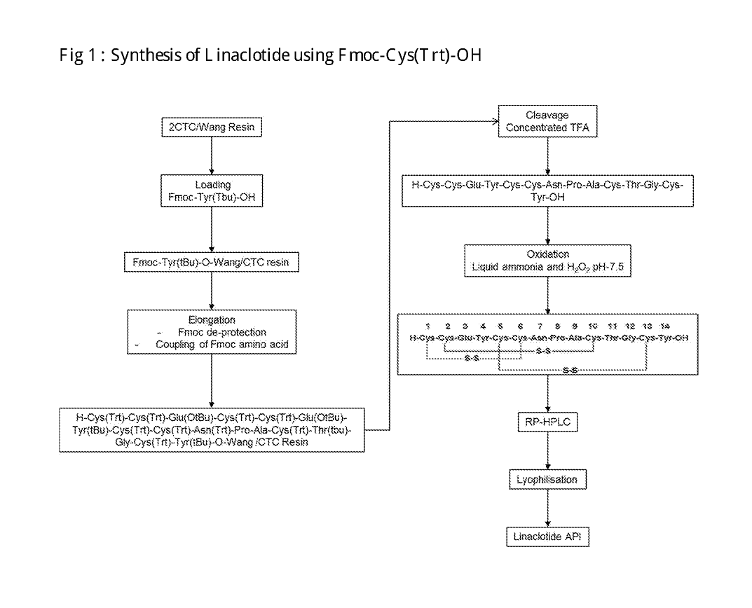

FIG. 2: Synthesis of Linaclotide using Fmoc-Cys(Phacm)-OH, by elongation of peptide with sequential addition of amino acids on solid phase peptide synthesis.

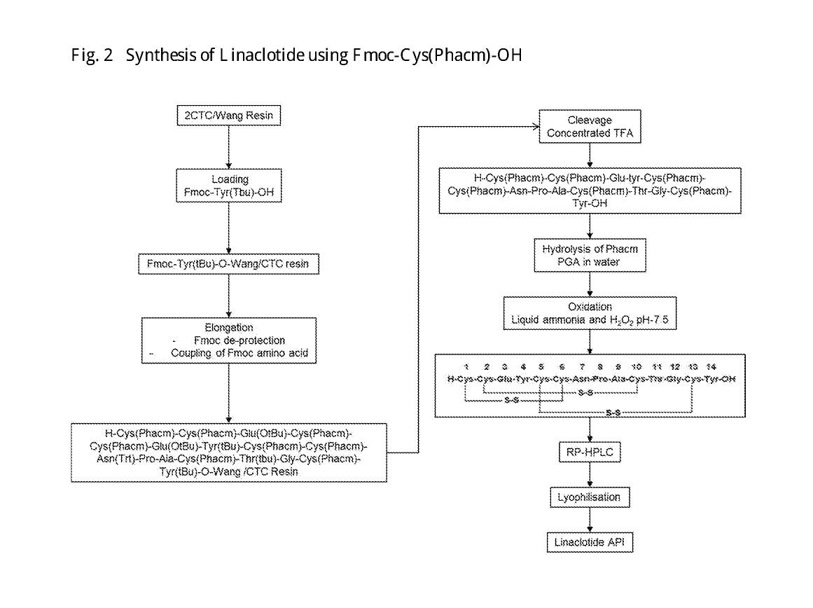

### ABBREVIATIONS

Fmoc ⁻Fluorenyl methyl oxycarbonyl
Boc—tert-butyloxycarbonyl
Bpoc—2-(4-Biphenylyl)-prop-2-yl 4-methoxycarbonyl phenyl carbonate
tBu—tert-butyl
cBz—carboxy benzyl
TFA—trifluoroacetic acid
EDT—ethanedithiol
TIS—triisopropylsilane

### DETAILED DESCRIPTION OF THE INVENTION

The present invention relates to a process for the preparation of Linaclotide by elongation of suitable protected fragments in presence of a coupling agent, simultaneous cleavage and de-protection of peptide, oxidation and isolation of Linaclotide.

Accordingly, in a preferred embodiment, the present invention provides a process for preparing Linaclotide of formula I,

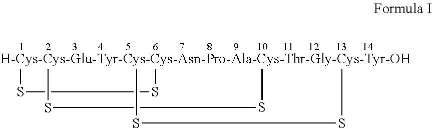

the said process comprising;
a) elongation of a peptide with sequential addition of amino acids by solid phase synthesis to obtain a protected peptide resin;
b) cleaving and deprotecting the resin simultaneously to obtain the linear Linaclotide of Formula (II);

```
          1 2 3 4 5 6 7 8 9 10 11 12 13 14
H-Cys-Cys-Glu-Tyr-Cys-Cys-Asn-Pro-Ala-Cys-Thr-Gly-Cys-Tyr-OH
                                                Formula (II)
```

c) oxidizing the linear Linaclotide of Formula (II) to obtain Linaclotide of Formula (I).

In an embodiment, peptide can be synthesized by the solid-phase synthesis. Solid Phase Peptide Synthesis (SPPS) can be defined as a process in which a peptide anchored by its C-terminus to an insoluble polymer is assembled by the successive addition of the protected amino acids constituting its sequence.

Each amino acid addition is referred to as a cycle consisting of:

a) cleavage of the N-protecting group

b) washing steps

c) coupling of a protected amino acid

d) washing steps

The formation of a peptide bond between two amino acids involves two steps. The first step is the activation of the carboxyl group of one residue. The second step is the nucleophilic attack of the amino group of the other amino acid derivative at the active carboxylic group.

The choice of an adequate combination of protecting groups to obtain a suitable amino acid chain is the first step on the way to achieve a successful peptide synthesis.

In an embodiment, for SPPS the protecting group is selected from the group consisting of traditional Fmoc/tBu protection or Boc/benzyl protection. Other protecting groups such as Cbz, Bpoc could also be used as amino protecting group.

In another preferred embodiment, the present invention provides for the preparation of protected peptide resin, the said process comprising,

a) cleavage of Fmoc ⁻group from F-moc protected amino acids to obtain fragments; and

b) coupling of fragments by a solid phase synthesis to obtain a protected peptide resin.

In an embodiment, Fmoc protected amino acid is linked to acid sensitive resins.

In an embodiment, the range of acid sensitive resins which can be used for the synthesis of (fully protected) peptide alcohols and thiols, is selected from the group consisting of Wang resin, Chlorotrityl Chloride (CTC) resin, Diphenyl diazomethane resin (PDDM-resin), Tricyclic amide linker resin, ‘Rink amide_ resins, 4,4\check{}-Dialkoxybenzhydrylamine resin, ‘PAL_ resin, 4-alkoxy-2,6-dimethoxy benzylamine resin, 4-methytrityl chloride, TentaGel S 25 and TentaGel TGA.

The esterification of Wang resin as well as of other resins such as CTC resin, bearing hydroxyl groups with Fmoc amino acids is a crucial step in SPPS.

In an embodiment, Wang resin, i.e. p-alkoxybenzyl alcohol resin, may be termed the standard resin for Fmoc/tBu SPPS of ‘peptide acids_. The tert butyl type side-chain protection is concomitantly removed during acidolytic cleavage from this resin.

In another embodiment, Chlorotrityl Chloride (CTC) resin can also be used for the solid-phase synthesis of C-terminal acid peptides. CTC resin can be used for the preparation of both protected and unprotected peptides. This forms one aspect of the present invention. Further, regeneration of the CTC resin after cleavage of the target compounds is also feasible, which allows the reuse of the resin. This forms another aspect of the present invention.

Alternatively preloaded resins, which are Fmoc L- or D-amino acids coupled to Wang/CTC resin can also be used.

In a preferred embodiment, the CTC resin reacts with Fmoc amino alcohols in the presence of pyridine(or DIPEA/DMAP) in DCM/DMF to obtain Fmoc-Tyr (tBu)-CTC resin. The resulting ethers can be cleaved by mild acid.

The coupling reactions are generally faster in SPPS than in solution, thus minimizing loss of configuration.

At the beginning of each elongation cycle, deblocking or washing step, the resin and the solution are mixed thoroughly, for the remaining process.

The most-widely used coupling reagents are carbodiimides on one hand, and phosphonium and aminium salts on the other hand.

In an embodiment of the present invention the coupling agents are selected from the group comprising of N-hydroxysuccinimide (HOSu), 2-(1H-Benzotriazole-1-yl)-1,1,3,3-tetramethyluronium tetrafluoroborate (TBTU), dicyclohexylcarbodiimide (DCC), N,N0-diisopropylcarbodiimide (DIC), N-hydroxytetrazole (Hot), ethyl 1-hydroxy-1H-1,2,3-triazole-4-carboxylate (HOCt), 2-(1H-benzotriazol-1-yl)-1,1,3,3-tetramethyluroniumhexaZuoro phosphate (HBTU), Benzotriazol-1-yl-oxy-tris(dimethylamino) phosphonium hexafluorophosphate (BOP), Benzotriazol-1-yloxy tri(pyrrolidino)phosphonium hexafluorophosphate (PyBOP), Bromotri (pyrrolidino)phosphonium hexafluorophosphate (PyBrOP), Chlorotri(pyrrolidino)phosphonium hexafluorophosphate (PyClOP), Pure, 0-(6-Chlorobenzotriazol-1-yl)-1, 1,3,3-tetramethyl uronium tetrafluoroborate (TCTU), Ethyl 1,2-dihydro-2-ethoxyquinoline-1-carboxylate (EEDQ), 1-Cyano-2-ethoxy-2-oxoethylidenaminooxy) dimethylaminomorpholino carbenium hexafluorophosphate (COMU), 3-(Diethoxy-phosphoryloxy)-3H-benzo[d][1, 2,3]triazin-4-one (DEPBT), 1-hydroxy-1,2,3-triazole derivatives, Ethyl cyano(hydroxyimino)acetate (Oxyma Pure) and the like, and mixtures thereof.

In an embodiment, coupling is performed in the presence of an additive. An additive reduces loss of configuration at the carboxylic acid residue, enhances coupling rates and reduces the risk of racemization. The additive used in the process of the present invention is selected from, 1-oxo-2-hydroxy dihydrobenzotriazine (HODhbt), 1-hydroxybenzotriazole (HOBt), 7-aza-1-hydroxybenzotriazole (HOAt), 6-CF3-HOBt, 6-NO2-HOBt, ethyl-2-cyano-2-(hydroxyimino) acetate (Oxyma) and the like, or mixtures thereof.

In another embodiment of the present invention the coupling takes place in one of the solvents selected from the group comprising of DMF, DCM, THF, NMP, DMA or mixtures thereof,

In an embodiment Fmoc group is first cleaved from Fmoc-Tyr (tBu)-CTC resin by treating with a suitable secondary base piperidine, pyrrolidine, derivatives of piperadine in DMF followed by washing with DMF to obtain H-Tyr (tBu)-CTC. The elongation by coupling with next protected amino acid is then performed using suitable coupling reagent, additive and solvent.

In a preferred embodiment coupling is done sequentially, by a solid phase synthesis using DIPCDI (DIC) as a coupling agent and HOBT as an additive, to obtain side chain protected peptide.

In an embodiment-side chain functional groups of amino acids can be protected using appropriate protecting groups to avoid side reactions, during synthesis and cleavage. For example, the following protecting groups can be used: t-butyloxycarbonyl (alpha-amino groups); acetamidomethyl (thiol groups of Cys residues); 4-methylbenzyl (thiol groups of Cys); benzyl (y-carboxyl of glutamic acid and the hydroxyl group of threonine); and bromobenzyl (phenolic group of tyrosine,).

In an embodiment, Cysteine thiol protecting groups used in the coupling reactions are either trityl protected or S-Phenylacetamidomethyl (Phacm) protected.

The Phacm group has the same stability and orthogonality as acetamidomethyl (Acm) and has the additional advantage that it can be deprotected simply by water in the presence of the enzyme penicillin amidohydrolase (PGA). Thus, Phacm is orthogonal with the common cysteine-protecting groups, such as 4-methylbenzyl (p-MeBzl), trityl (Trt) and fluorenylmethyl (Fm). This forms one aspect of the present invention.

Removing Phacm under the mild (using water) and highly specific conditions (using PGA enzyme) results in formation of Linaclotide in high yield without any adduct. This forms another aspect of the present invention.

In an embodiment, the protected peptide is cleaved from the peptide resin and deprotected, simultaneously to obtain linear Linaclotide.

Prior art WO 2015/022575, suggests concurrent cleaving the protected peptide from the peptide resin and deprotecting the peptide to obtain linear Linaclotide.

Thus the prior art suggest two steps synthesis of linear Linaclotide using two different deprotecting agents;

a) cleavage of protected peptide from the peptide resin in DCM using TAF, followed by b) deprotection of protected linear Linaclotide using TAF-TIS-water-EDT.

Whereas, in the present invention, the inventors have provided a one step process to obtain a linear Linaclotide. This forms one aspect of the present invention. The simultaneous cleavage avoids use of two different deprotecting agents and handling of large quantity of solvents. This forms another aspect of the present invention.

Further, the process of the present invention avoids isolation of linear Linaclotide solids as reported in the prior art by neutralization, extraction, concentration and isolation performed at each stage. This forms yet another aspect of the present invention.

In an embodiment cleavage is performed in concentrated TFA and optionally in presence of scavengers.

Some of the amino acid side chains are sensitive to chemical reactions and the carbocations groups liberated are very reactive. They have to be trapped as to avoid undesired reactions with sensitive amino acids such as Cys, Thr, Tyr. Scavengers are used to surround the amino acid side chain protecting groups and prevent them from reattaching to the peptide.

In an embodiment, water is a moderately efficient scavenger and can be used as single scavenger for the cleavage of peptides devoid of Cys. Whereas EDT and DTE are often-used and efficient scavengers for peptides containing sensitive amino acids. Alternatively, the cleavage cocktail/combination comprising scavenger reagents may be used.

Before performing the cleavage, the resin is thoroughly washed with DCM to remove all traces of DMF. Residual basic DMF can inhibit TFA acidolysis. The peptide resin is dried under high vacuum for at least 4 h, or preferably over night over KOH.

The cleavage is preferably performed in ‘K_ reagent TFA/Thioanisol/Phenol/Water/ethandithiol using (82.5:5:5:5:2.5) ratio (v/v/v/v/w) at RT for >K hr to 4 hrs.

Silane derivatives triethylsilane (TES), triisopropylsilane (TIS) can successfully replace the malodorous EDT as well. Alternatively cleavage may be performed in TFA/TIS/Water using 95:2.5:2.5 ratio (v/v/v/w) at RT for >K hr to 4 hrs.

In yet an alternative embodiment cleavage may be performed in the presence of combination of solvents in specific ratios selected from the group consisting of TFA/TIS/water (92.5:5:2.5)(v/v/v); TFA/TIS/H₂O (95:2.5:2.5, v/v/v) TFE/AcOH/DCM (1:1:3); TFA:Phenol:H₂O:TIS (88:5:5:2, v/v/v/v); TFA:TIS:DTT:H₂O (88:2:5:5, v/v/v); 0.5% TFA/DCM; HFIP/DCM (1:4 or 3:7, v/v); TFA:TIS:DCM (25:15:60, v/v/v).

In an embodiment, the amount of TFA used in the cocktails, varies from 15% to 95%.

Preferably, cleavage is performed using cleavage cocktail TFA:TIS:DCM (25:15:60, v/v/v). The hydrophobic impurities found with [symbol] K\check{} Reagent also reduced, thereby increasing the overall purity >70%.

After completion of the reaction, the solid is isolated by simple precipitation using counter-solvent such as MTBE, DIPEA or water, filtered and dried to afford linear Linaclotide-TFA, thus making it economical and suitable on industrial scale. This forms yet another aspect of the present invention.

In an embodiment, when the Cysteine thiol protecting groups used is other than Phacm; the linear Linaclotide-TFA thus obtained is in a free thiol form.

When the Cysteine thiol protecting groups used is Phacm the obtained solid is linear linaclotide-TFA in a protected thiol form.

In an embodiment of the present invention, linear linaclotide-TFA is further oxidized using suitable oxidizing reagent.

In an embodiment, oxidation by molecular oxygen or other appropriate oxidizing reagents provide the intramolecular di sulfide to obtain linaclotide.

In an embodiment, the oxidizing agent is selected from a group comprising of hydrogen peroxide, dimethyl sulfoxide (DMSO), glutathione, and the like, and a mixture thereof.

In yet another embodiment, of the present invention, the oxidation is carried out at a pH range of about 7 to about 10.

In yet another embodiment of the present invention, the oxidation is carried out optionally in a buffer or an aqueous base such as liq. Ammonia.

In yet another embodiment of the present invention, the buffer is selected from a group comprising of ammonium acetate, sodium carbonate, ammonium bicarbonate,—and the like and a mixture thereof.

In an embodiment, molecular oxygen is used to promote disulfide formation under alkaline conditions e.g. pH 7.5 to 8.5, by simple aeration under gentle stirring or slow bubbling, thro\check{} dilute peptide solution. Preferably, water in the concentration of 0.2 mg of the reactant/ml is used for cyclization, which is completed (RT, pH 8-10) normally within 15 h to 25 h.

The addition of co-solvents like acetonitrile, DMSO helps to improve the solubility of hydrophobic sequences.

In yet another embodiment, when linear Linaclotide-TFA is in the protected thiol form, water and immobilized PGA in combination with DMSO in the ratio (80:20) make hydrolysis and cyclization a one pot reaction, which is completed (376éC, pH 7) normally within 15 h to 25 h.

After completion of the reaction, by simple filtration process, PGA can be removed from the reaction mixture and reused many times, this forms one aspect of the present invention.

Through the mild and highly specific conditions removing Phacm with PGA no adduct formation occurs and the desired cyclic peptide is being formed in high yield. This forms another aspect of the present invention.

Further, as the growing chain is bound to an insoluble support the excess of reagents Phacm and soluble by-products can be removed by simple filtration.

In yet an alternative embodiment, the present invention provides an improved process for preparing disulfide bridges of Linaclotide of Formula (I). The process comprises, reacting linear chain Linaclotide of Formula (II)

```
          1 2 3 4 5 6 7 8 9 10 11 12 13 14
H-Cys-Cys-Glu-Tyr-Cys-Cys-Asn-Pro-Ala-Cys-Thr-Gly-Cys-Tyr-OH
                                                Formula (II)
```

without use of an oxidizing agent to prepare appropriate bridges of Linaclotide of Formula (I)

Due to the poor solubility of linear chain Linaclotide, the prior art documents suggest oxidation in aqueous buffer systems at very low product concentrations.

Prior art CN105017387 teaches stepwise formation of disulfide linkages by oxidation of linear chain Linaclotide using a concentration of 5 mg/ml. Whereas WO2016/012938 teaches oxidation at a concentration of 0.1-0.2 mg/ml to 14 mg/ml. The oxidation at such a lower concentration hugely increases load volumes on further chromatography steps. The crude Linaclotide or the reaction mixture containing Linaclotide is purified by either anion or cation exchange chromatography. The Linaclotide having purity more than 80% obtained by either anion or cation exchange chromatography is further purified by another anion or cation exchange chromatography. The Linaclotide having purity more than 90% is further purified by reverse phase chromatography and lyophilized to get HPLC purity more than 99%. The crude Linaclotide or the reaction mixture containing Linaclotide may be purified by hydrophobic interaction, and may be further purified by another hydrophobic interaction and/or reverse-phase chromatography.

Thus the aforesaid prior art disclosures suggest multiple steps chromatography to obtain pure Linaclotide.

As compared to the prior art, the process of the present invention is advantageous as linear Linaclotide is oxidized at a higher concentration.

Any of the prior art processes, for example the WO2015/022575 process or 3858/MUM/2016 process, for preparing linear Linaclotide of formula (II) may be used.

In an embodiment oxidation is carried out without use of oxidizing agents such as dimethyl sulfoxide, hydrogen peroxide and the like.

In an embodiment, oxidation by molecular oxygen provides the intramolecular disulfide to obtain Linaclotide.

In yet another embodiment, of the present invention, oxidation is carried out at a pH range of about 7 to about 10.

In an embodiment, molecular oxygen is used to promote disulfide formation under alkaline conditions e.g. pH 7.5 to 8.5, by simple aeration under gentle stirring or slow bubbling, through concentrated peptide solution.

Preferably, water or aqueous solvent in the concentration of about 20 mg of the reactant/ml is used for cyclization. More preferably water or aqueous solvent in the concentration of 30 mg of the reactant/ml is used for cyclization.

The aqueous solvent used may be selected form alcoholic solvents such as methanol, ethanol or isopropanol, aprotic solvent such as acetonitrile and the like. Preferably solvent used is ethanol. The solvent to the water ratio may vary from 40:60 v/v to 60:40 v/v. Preferably, solvent to the water ratio used is 50:50 v/v. More preferably, the solvent to water ratio is 55:45 v/v.

The reaction mixture is stirred at about 0éC to about 30éC for about 1 h to about 40 h. Particularly, the reaction mixture is stirred at about 20éC to about 30éC for about 5 hours to about 30 hours. More particularly, the reaction mixture is stirred at about 20éC to about 25éC for about 6 hours to about 15 hours. The purity of Linaclotide at this stage is about 40-50% by HPLC.

It is observed that, the non-polar impurities i.e. high molecular weight impurities (such as dimers, trimers & the likes) precipitate out when the reaction mixture is stirred under acidic conditions at low temperature. Hence, after completion of oxidation, the reaction mixture is further acidified to pH 2-4 using suitable acid such as glacial acetic acid, trifluoro acetic acid and the like and stored at about 0éC to about 10éC for about 0.5 h to about 3 h. The impurities are precipitated, which are removed either by centrifugation or filtration. The Linaclotide having HPLC purity of more than 70% may be obtained at this stage by the process of the present invention.

The prior art processes disclosed in the citations listed above, suggest formation of crude Linaclotide having HPLC purity of more than about 70% at an initial concentration of about 2 mg/ml. Whereas crude Linaclotide having HPLC purity of more than about 70% at an initial concentration of about 30 mg/ml, in the given reaction mixture, is formed by the process of the present invention. This forms one aspect of the present invention.

The higher reaction concentration reduces solvent volume by at least 25 to 50 folds as compared to the prior art process. Thus, the present process avoids handling of large quantity of solvent on industrial scale and this forms another aspect of the present invention.

The reaction mixture containing crude Linaclotide is further diluted with water to reduce the percentage of the solvent in the reaction mass. Particularly, the solvent percentage is reduced up to 10% v/v.

The excess acid may be present in the reaction mass after reaction completion, which may be neutralized by adjusting the pH to 7 to 9 using liquid ammonia.

The crude Linaclotide or a reaction mixture containing Linaclotide is further purified by successive Reverse Phase Chromatography (RPC), prior to lyophilization. The column which may be used for the reverse-phase chromatography may be any known column employed in the art. In one embodiment, the column may be YMC Triart C-18, 10 [symbol] column.

In another embodiment, the column may be a Kromasil C18 column or Daiso C-18, 10 [symbol] or Phenomenex Luna C18 (2) column.

In an embodiment of the present invention, the Linaclotide obtained by the process of the present invention is subjected to the successive Reverse Phase Chromatography (RPC) prior to lyophilization.

RPC takes advantage of hydrophobic interactions between the stationary phase and the mobile phase for purification. Polar impurities elute faster than the hydrophobic ones. In an embodiment, RPC is combined with appropriate mobile phase to desalt the compound and help purify enantiomers.

In an embodiment, the present invention provides a substantially pure Linaclotide of formula (I) free from dimer and multimer impurities. As used herein, substantially pure refers to chemical and optical purity of greater than about 97%, preferably greater than about 98%, and greater than about preferably 99.0% by weight.

In another embodiment, the present invention provides Linaclotide prepared according to the process of the present invention for use in treating irritable bowel syndrome and chronic constipation.

Accordingly, there is provided herein a method for treating irritable bowel syndrome and chronic constipation thereof, the method comprising administering to a subject in need thereof a therapeutically effective amount of Linaclotide prepared according to the process of the present invention.

While emphasis has been placed herein on the specific steps of the preferred process, it will be appreciated that many steps can be made and that many changes can be made in the preferred steps without departing from the principles of the invention. These and other changes in the preferred steps of the invention will be apparent to those skilled in the art from the disclosure herein, whereby it is to be distinctly understood that the foregoing descriptive matter is to be interpreted merely as illustrative of the invention and not as a limitation.

The details of the invention given in the examples which are provided below are for illustration only and therefore these examples should not be construed to limit the scope of the invention.

### EXAMPLES

### Example 1. Synthesis of Linaclotide Using Trt Protected Cys Amino Acids

A) Synthesis of Fmoc-Tyr(tbu)-CTC Resin
   CTC resin (5 g, 8 mmol, 1.6 mmol/g) was placed into solid phase reactor. Mixture of Fmoc-tyr(tBu)-OH(7.4 g, 16 mmol, 2 equivalent) And DIPEA (4.2 ml, 9 equiv) is prepared in DCM (24 ml) and prepared mixture was added in the solid phase Reactor. Reaction was stirred for 2 hrs at RT. After reaction was complete, the resin was capped by adding DCM (42.5 ml), DIPEA(2.5 ml), MeOH (5 ml) for 30 min at RT then Resin was washed with DMF (5B50 ml) and DCM (3B 50 ml) and dried.
   Yield: 6.58 g
B) Synthesis of H-cys(Trt)-Cys(Trt)-Glu(tBu)-Tyr(tBu)-Cys(Trt)-Cys(Trt)-Asn(Trt)-Pro-Ala-Cys(Trt)-Thr(tBu)-Gly-Cys(trt)-Tyr(tBu)-CTC Resin Cleavage of Fmoc group was effected by treating Fmoc-Tyr(tbu)-CTC Resin (3 g) with 20% (V/V) Piperidine in DMF (2B 30 ml) 2 min and 10 min Respectively, followed by washing resin with DMF (5B30 ml) 2 min each. Fmoc-Cys(Trt)-OH(6.38 g, 3 equiv), HOBT monohydrate(1.67 g, 3 equiv) and DIPCDI (1.37 ml, 3 equiv) were dissolved in DMF(15 ml) stir for 1 min, added to the resin, and coupling reaction was stirred for 1 h. After reaction was complete, the resin was washed with DMF (5B30 ml). By following this procedure, the following Fmoc-Amino acids (AAs) (3 equiv) were sequentially incorporated with DIPCDI (3 equiv) and HOBt monohydrate (3 equiv) in DMF. Cleavage of Fmoc group was effected by treatment with 20% (v/v) piperidine in DMF (2B 30 ml) 2 min and 10 min each. (Note: AAs=Fmoc-Cys(Trt)-OH, Fmoc-Glu(otBu)-OH, Fmoc-Tyr(tBu)-OH, Fmoc-Ala-OH, Fmoc-Pro-OH, Fmoc-Thr(tBu)-OH, Fmoc-As(Trt)-OH)
   Yield: 12 g
C) Synthesis of H-Cys-Cys-Glu-Tyr-Cys-Cys-Asn-Pro-Ala-Cys-Thr-Gly-Cys-Tyr-OH.TFA(Crude Linear Linaclotide-TFA)
   After finishing the elongation, side chain protected peptide resin (12 g) was treated with the reagent K (TFA-Thioanisol-Phenol-Water-EDT) (82.5:5:5:5:2.5) 110 ml at RT for 3 h under stirring. After reaction was complete, reaction mass was filtered. The filtrate was then precipitated with MTBE (420 ml), filtered and washed with MTBE (5B75 ml). The filter cake was dried in vacuum at 30éC to obtain crude linear Linaclotide-TFA.
   Yield: 3.45 g
   Purity of Linear Linaclotide-66.96%
   Observed mass—1533.8
D) Synthesis of Crude Linaclotide
   The crude linear Linaclotide-TFA (250 mg) was subjected to Air oxidation in water (1250 ml) pH of reaction was maintained 8-9 with liquid ammonia. Reaction was carried out for 24 hr at RT.
   Purity: 60%
   The crude peptide was then subjected to purification on RP ⁻Chromatography followed by lyophilization to give pure Linaclotide.

### Example 2. Synthesis of Linaclotide Using Phacm Protected Cys Amino Acids

A) Synthesis of Fmoc-Tyr(tbu)-CTC Resin
   CTC resin (5 g, 8 mmol, 1.6 mmol/g) was placed into solid phase reactor. Mixture of Fmoc-tyr(tBu)-OH(7.4 g, 16 mmol, 2 equivalent) and DIPEA (4.2 ml, 9 equiv) is prepared in DCM (24 ml) and the prepared mixture was added in the solid phase Reactor. Reaction was stirred for 2 hrs at RT. After reaction was complete, the resin was capped by adding DCM (42.5 ml), DIPEA(2.5 ml), MeOH (5 ml) for 30 min at RT then Resin was washed with DMF (5B50 ml) and DCM (3B 50 ml)
   Yield: 6.5 g
B) Synthesis of H-cys(Phacm)-Cys(Phacm)-Glu(tBu)-Tyr(tBu)-Cys(Phacm)-Cys(Phacm)-Asn(Trt)-Pro-Ala-Cys(Phacm)-Thr(tBu)-Gly-Cys(Phacm)-Tyr(tBu)-CTC Resin
   Cleavage of Fmoc group was effected by treating Fmoc-Tyr(tbu)-CTC Resin(3 g) with 20% (V/V) Piperidine in DMF (2B 30 ml) 2 min and 10 min Respectively, followed by washing resin with DMF (5B30 ml) 2 min each. Fmoc-Cys (Phacm)-OH(5.34 g, 3 equiv), HOBT monohydrate(1.67 g, 3 equiv) and DIPCDI (1.37 ml, 3 equiv) were dissolved in DMF(15 ml) stir for 1 min, added to the resin, and coupling reaction was stirred for 1 h. After reaction was complete, the resin was washed with DMF (5B30 ml). By following this procedure, the following Fmoc-AAs (3 equiv) were sequentially incorporated with DIPCDI (3 equiv) and HOBt monohydrate(3 equiv) in DMF. Cleavage of Fmoc group was effected by treatment with 20% (v/v) piperidine in DMF (2B 30 ml) 2 min and 10 min each. (Note: AAs=Fmoc-Cys(Phacm)-OH, Fmoc-Glu(otBu)-OH, Fmoc-Tyr(tBu)-OH, Fmoc-Ala-OH, Fmoc-Pro-OH, Fmoc-Thr(tBu)-OH, Fmoc-As(Trt)-OH) Yield: 9.71 g
C) Synthesis of H-Cys(Phacm)-Cys(Phacm)-Glu-Tyr-Cys(Phacm)-Cys(Phacm)-Asn-Pro-Ala-Cys(Phacm)-Thr-Gly-Cys(Phacm)-Tyr-OH.TFA (Linear S-Phacm-protected Linaclotide-TFA)
   After finishing the elongation, side chain protected peptide resin was treated with the TFA-TIS-Water (95:2.5:2.5) at RT for 3 h under stirring. After reaction was complete, reaction mass was filtered. The filtrate was then precipitated with MTBE (420 ml), filtered and washed with MTBE (5B75 ml). The filter cake was dried in vacuum at 30éC to obtain Linear S-Phacm-protected Linaclotide-TFA.
   Yield: 575 mg,
   Purity: 43.47%
   Observed mass: 2416.8
D) Synthesis of crude Linaclotide:
   Linear S′ Phacm-Protected Linaclotide (x g, 0.1 mg/ml) was dissolved in mixture of water/DMSO (95:5, X ml), immobilized PGA was added and reaction was left to stand for 24 h at RT and 500 RPM. Immobilized biocatalyst was removed by filtration from media and crude peptide subjected to RP Chromatography followed by lyophilization to give pure Linaclotide.

### Example 3. Synthesis of Linaclotide

Linear Linaclotide (285 mg) was dissolved in a mixture of ethanol-water (1:1) (9.5 ml). The pH of the reaction mass was adjusted to 7-9 using liquid ammonia. This reaction mass was stirred at 25-30é C for about 6 h to about 9 h. The pH of the reaction mass was adjusted to 6-7 using acetic acid.

HPLC purity˜40-50%.

The reaction mass was further acidified to pH 2-4 using glacial acetic acid and stored at 2-8éC for about 1 h. The reaction mass was filtered.

HPLC purity ˜60-70%

The reaction mixture was further diluted with water (38 ml) and the pH was adjusted to 7-9 using liquid ammonia. The crude peptide subjected to RP Chromatography. The pure Linaclotide was eluted using acetonitrile and 0.5% acetic acid gradient. Fractions with purity more that 98% were pooled, distilled and lyophilized to get Linaclotide as a white amorphous powder.

HPLC purity >99.0%

Yield >50%.

### Example 4. Two Steps Synthesis of Linear Linaclotide ⁻TFA (Prior Art Process)

Step 1: Cleavage from Resin

The side chains protected Linaclotide (1 g) was cleaved from resin using 1% TFA in DCM (5 B 80 ml B 10 min) at 0éC, the protected peptide was then washed with DC M (5 B 80 ml B 10 min), concentrated and extracted three times with 1% Acetic Acid and Ethanol. The organic layer was concentrated, precipitated with heptane (100 ml) under stirring for 2 h. The filter cake containing protected peptide was washed with heptane (3 B 80 ml) and dried for 24 h at 30ιC.

Step 2: Synthesis of H-Cys-Cys-Glu-Tyr-Cys-Cys-Asn-Pro-Ala-Cys-Thr-Gly-Cys-Tyr-OH.TFA (Crude Linear Linaclotide-TFA)

The protected peptide was treated with a mixture of TFA:TIS:H₂O (80:15:5) (30 ml), for 3 h and then toluene (156 ml) was added. Toluene layer was separated and the aqueous layer was precipitated using DIPEA (150 ml). Filtered and washed with DIPEA (3 B 50 ml). The filter cake was dried in vacuum at 30ιC to obtain crude linear Linaclotide-TFA.

HPLC purity—43.63%

### Example 5. One Step Synthesis of Linear Linaclotide ⁻TFA Using Cocktail

Different compositions were chosen in order to increase the purity by reducing the number steps from 2 to 1. Linear Linaclotide (1 g) was cleaved from the resin using different cocktails as listed in Table. 1 and stirred for 4 h at 25-30éC. Reaction mass was filtered through sintered funnel and resin was washed with TFA (5 ml). Filtrate was then poured on to chilled MTBE slowly and stirred for 30 min. Crude Linear Linaclotide was precipitated out, filtered through Sintered funnel and wash with chilled MTBE (5×5 ml). The filter cake was dried in vacuum at 30éC to obtain crude linear Linaclotide-TFA. Purity obtained by using different cocktails is as per Table. 1.

TABLE 1
Different cleavage parameters

| S. | CLEAVAGE COCKTAIL | | PURITY |
|---|---|---|---|
| NO | COMPONENTS | COMPOSITION | OBTAINED |
| 1 | TFA:TIS:H₂O | 95:2.5:2.5 | 7.66% |
| 2 | TFA:Phenol:H₂O:TIS | 88:5:5:2 | 8.49% |
| 3 | TFA:TIS:DTT:H₂O | 88:2:5:5 | 45.09% |
| 4 | :K ̆Reagent- | 82.5:5:2.5:5:5 | 60.85% |
| | TFA:Thioanisole: | | |
| | Ethanedithiol:H₂O:Phenol | | |
| 5 | TFA:TIS:DCM | 25:15:60 | 73.33% |

### Example 6. Synthesis of Linaclotide

Linaclotide of Formula I was produced using increasing concentration of linear Linaclotide in solvent system containing water. For this, Linear Linaclotide (Formula II) was stirred in water. The pH of the reaction mass was adjusted to 9 to 9.5 using liquid ammonia. This reaction mass was stirred at 25-30é C for about 18 h. The pH of the reaction mass was adjusted to 6-7 using acetic acid.

Examples for different concentrations of linear Linaclotide are given in the following table 2.

TABLE 2
Oxidation with increasing linear L inaclotide concentration

| | | LINEAR | | | |
|---|---|---|---|---|---|
| | | LINACLOTIDE | | | |
| S. | | CONCENTRATION | | Time | |
| NO. | Solvent | (w/v) | Temp | (Hrs) | PURITY |
| 1 | water | 0.2 mg/ml | 25ĕ2 [symbol] | 18 Hrs | 54.45% |
| 2 | water | 0.5 mg/ml | 25ĕ2 [symbol] | 18 Hrs | 32.82% |
| 3 | water | 1 mg/ml | 25ĕ2 [symbol] | 18 Hrs | 39.05% |
| 4 | water | 1.5 mg/ml | 25ĕ2 [symbol] | 18 Hrs | 37.56% |
| 5 | water | 2 mg/ml | 25ĕ2 [symbol] | 18 Hrs | 29.53% |
| 6 | 5% DMSO | 0.2 mg/ml | 25ĕ2 [symbol] | 6 Hrs | 50.84% |
| | in water | | | | |

### Example 7. Synthesis of Linaclotide

Linaclotide of Formula I was produced using mixture of organic solvents containing water. For this, Linear Linaclotide (Formula II) at a concentration of 20 mg/ml (w/v) was stirred in a mixture of organic solvents in water. The pH of the reaction mass was adjusted to 8 to 9 using liquid ammonia. This reaction mass was stirred at 25-30éC. The pH of the reaction mass was adjusted to 6-7 using acetic acid.

Examples for different concentrations of the components of the solvent system are given in the following table 3.

TABLE 3
Oxidation with different solvent system at 20 mg/ml W/V

| S. | SOLVENT | TIME | | | |
|---|---|---|---|---|---|
| NO. | SYSTEM | (h) | Temp | PURITY | CONVERSION |
| 1 | IPA:Water (60:40) | 24 | 25ĕ2 [symbol] | 79.76% | 4.61% |
| 2 | Water | 12 | 25ĕ2 [symbol] | 45.75% | 16% |
| 3 | ACN:Water (60:40) | 24 | 25ĕ2 [symbol] | 24.47% | 44.99% |
| 4 | MeOH:Water | 6 | 25ĕ2 [symbol] | 39.93% | 69% |
| | (60:40) | | | | |
| 5 | 20% DMSO | 12 | 25ĕ2 [symbol] | 52.65% | 80% |
| | Ethanol:Water | | | | |
| | (55:45) | | | | |
| 6 | Ethanol:Water | 12 | 25ĕ2 [symbol] | 57.35% | 84% |
| | (55:45) | | | | |
| 7 | Ethanol:Water | 24 | 25ĕ2 [symbol] | 79.08% | 85% |
| | (55:45) | | | | |
| 8 | Ethanol:Water | 24 | 25ĕ2 [symbol] | 79.46% | 84% |
| | (70:30) | | | | |

### Example 8. Synthesis of Linaclotide

Linaclotide of Formula I was produced using increasing product concentration in a mixture of ethanol:water (55:45). For this, Linear Linaclotide with increased concentration was stirred in a mixture of ethanol:water (55:45). The pH of the reaction mass was adjusted to 9 to 9.5 using liquid ammonia. This reaction mass was stirred at 25é-30é C for about 24 h. The pH of the reaction mass was adjusted to 6-7 using acetic acid.

Examples for different concentrations of linear Linaclotide subjected to oxidation are given in the following table 4.

TABLE 4
Oxidation in EtOH:Water (55:45) with different product
concentration

| S. | LINEAR LINACLOTIDE | | | |
|---|---|---|---|---|
| NO. | CONCENTRATION | PURITY | Temp | CONVERSION |
| 1 | 20 mg/ml | 79.08% | 25ĕ2 [symbol] | 85% |
| 2 | 40 mg/ml | 77.2% | 25ĕ2 [symbol] | 49% |
| 3 | 80 mg/ml | 72.11% | 25ĕ2 [symbol] | 23% |
| 4 | 200 mg/ml | 9.5% | 25ĕ2 [symbol] | 1% |

### Example 9. Synthesis of Linaclotide

1.15 g of Linear Linaclotide was stirred at room temperature in a mixture of ethanol:water (55:45 v/v, 80 ml). The pH of the reaction was adjusted to 8 by using 10% Liq. Ammonia solution (3.2 ml). The reaction mass was stirred for 12 hrs at 25-30éC and quenched by reducing pH to 6 using 1 ml glacial acetic acid. Table. 5. % conversion at various time intervals

| Time (Hrs) | % purity | % Conversion |
|---|---|---|
| 3 | 15.72% | 20% |
| 6 | 49.94% | 70% |
| 9 | 54.98% | 85% |
| 12 | 57.36% | 84% |
| Quenched | 69.17% | 83% |

Yield: −332 mg

Efficiency: −83.2%

Purification of crude Linaclotide

The oxidized product was purified by reverse phase HPLC and lyophilized to give pure Linaclotide. HPLC purity >99%

Sequence listing:

Seq ID No. 1:
<210> 1
<211> 14 amino acids
<212> Polypeptide
<213> Artificial sequence
<220> Linaclotide
<400> 1
Cys-Cys-Glu⁻Tyr⁻Cys⁻Cys⁻Asn⁻Pro⁻Ala⁻Cys⁻Thr⁻Gly⁻
Cys⁻Tyr

We claim:

1) A process for the preparation of Linaclotide of Formula I, 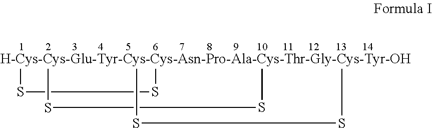
the said process comprising;
a) elongation of a peptide with sequential addition of amino acids by solid phase synthesis to obtain a protected peptide resin;
b) cleaving and deprotecting the resin simultaneously to obtain the linear Linaclotide of Formula (II);
```
          1 2 3 4 5 6 7 8 9 10 11 12 13 14
H-Cys-Cys-Glu-Tyr-Cys-Cys-Asn-Pro-Ala-Cys-Thr-Gly-Cys-Tyr-OH
                                                Formula (II)
```
c) oxidizing the linear Linaclotide of Formula (II) to obtain Linaclotide of Formula (I).

2) The process according to claim 1, wherein the amino acids used in step (a) are protected with protecting group selected from Fmoc/tBu, Fmoc/trt, Boc/benzyl, Cbz, Bpoc, Fmoc/Phacm or mixture thereof.

3) The process according to claim 1 or 2, wherein Fmoc protected amino acid is linked to acid sensitive resins selected from Wang resin, Chlorotrityl Chloride (CTC) resin, Diphenyldiazomethane resin (PDDM-resin), Tricyclic amide linker resin, ‘Rink amide_ resins, 4,4\check{}-Dialkoxybenzhydrylamine resin, ‘PAL_ resin, 4-alkoxy-2,6-dimethoxybenzylamine resin, 4-methytrityl chloride, TentaGel S 25 or TentaGel TGA.

4) The process according to any of the preceding claims, for the preparation of protected peptide resin, which comprises the following steps, a) cleavage of Fmoc ⁻group from F-moc protected amino acids to obtain fragments; and b) coupling of fragments by a solid phase synthesis to obtain a protected peptide resin.

5) The process according to claim 4, wherein the coupling is carried out in presence of coupling agent(s).

6) The coupling agent (s) according to claim 5, is selected from the group comprising of HOSu, TBTU, DCC, Hot, HOCt, HBTU, PyBOP, PyBrOP, PyClOP, TCTU, EEDQ, COMU, BOP, DEPBT, DIC, and the like, and mixtures thereof.

7) The process according to claims 5 and 6, wherein the coupling is performed in the presence of an additive selected from the group comprising of HODhbt, HOBt, HOAt, and Ethyl cyano(hydroxyimino) acetate (Oxyma Pure).

8) The process according to claim 1, wherein the step (b) is carried out in the presence of a cleavage cocktail selected from K_ reagent (TFA/Thioanisol/Phenol/Water/ethandithiol), TFA/TIS/Water, TFA/TIS/water, TFA/TIS/H₂O, TFE/AcOH/DCM, TFA/Phenol/H₂O/TIS, TFA/TIS/DTT/H₂O, 0.5% TFA/DCM, HFIP/DCM and TFA/TIS/DCM.

9) The process according to claim 1, wherein oxidation in the step (c) is carried out without use of oxidizing agents.

10) The process according to claims 1 and 9, wherein the concentration of linear Linaclotide is more than 20 mg/ml.

11) The process according to claims 1, 9 and 10, wherein the oxidation is carried in the presence of an aqueous solvent.

12) The process of claim 11, wherein the aqueous solvent comprises an organic solvent selected from the group consisting of alcoholic solvents such as methanol, ethanol or isopropanol; aprotic solvent such as acetonitrile and the like.

13) The process according to claim 12, wherein the oxidation is carried using aqueous ethanol.

14) The process of claim 1, wherein Linaclotide is prepared by treating a linear chain of peptide of formula (II) with ethanol:water in 55:45 v/v.

15) The process of claim 1, wherein Linaclotide is prepared by treating a linear chain of peptide of formula (II) with ethanol:water in 55:45 v/v at pH from about 7 to about 10.

16) The process of claim 15, wherein the temperature is about 20éC to about 30 éC.

17) The process of claim 4, wherein the reaction time is from about 5 hours to about 30 hours.

18) The process according claim 1, further comprising purification of Linaclotide by Reverse Phase Chromatography (RPC) followed by lyophilization.

19) The process according to claim 18, wherein Linaclotide is having purity greater than 99%.

20) The process according to claim 18, wherein the Linaclotide is substantially free from dimer impurities and multimer impurities.
